(19) United States
(12) Patent Application Publication
(10) Pub. No.: US 2009/0073383 A1
(43) Pub. Date: Mar. 19, 2009

(54) Ophthalmoscope Utilizing a Stenopeic Aperture

(76) Inventors: Fernando Dantas Coutinho, Rio de Janeiro (BR); Marisa Marinho Dantas Coutinho, Rio de Janeiro (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/917,600

(22) PCT Filed: Jun. 16, 2005
(86) PCT No.: PCT/BR05/00111

§ 371 Date: Mar. 13, 2008

(51) Int. Cl. A61B 3/12

(52) U.S. Cl. 351/214

(57) ABSTRACT

An opthalmoscope comprising an attachment portion, capable of being clipped onto a commonly available light source, such as a penlight; a light deflector, such as a mirror or prism, for deflecting light from the light source into a subject eye; a housing, for holding the deflector in fixed relation to the attachment portion; and a stenopeic aperture, in the housing, between the light source and the deflector, for limiting the light incident on the deflector to a specified meridian.

### TECHNICAL FIELD

The present invention relates to an opthalmoscope that is cheap and simple to manufacture, and can be used for both direct and indirect ophthalmic observation of the eye.

### BACKGROUND ART

Opthalmoscopes are routinely used by medical practitioners, from general practitioners to ophthalmologists to veterinary surgeons. In general, the opthalmoscopes available commercially today are complex pieces of equipment, containing numerous expensive optical components and are therefore prohibitively expensive for most medical students and lay practitioners, especially in less developed countries. The availability of a cheap alternative to these expensive opthalmoscopes would also enable medical workers to diagnose and treat a number of diseases prophylactically.

A number of attempts have been made to provide cheap alternatives to these expensive opthalmoscopes, for example, patent application PCT/GB2002/005758 to Armour, describes a lens-free opthalmoscope which utilizes a penlight as a light source and a reflective means, contained in a hollow tube, for directing the light from the penlight through a first window in the tube. The hollow tube includes a second window, through which the subject eye is observed, and a baffle is included in the path between the light source and the second window in order to prevent extraneous light from interfering in the observation. While the opthalmoscope according to Armour does not include expensive optical components, it is not very easy to manufacture, especially on a large scale production basis, since it includes a number of components that require working and assembly. Also, the opthalmoscope according to Armour does not include treatment of aberrations inherent in the light beam from a penlight light source.

Another example of an attempt to provide a cheap alternative to the traditional expensive opthalmoscopes can be found in patent application PCT/GB2001/003785 to Lo. This document describes an ophthalmic device for the examination and detection of opacities in the ocular media, in which a reflecting means is located in a tubular housing having a first through-hole, through which it deflects light from a penlight towards the subject eye, and a second through-hole, through which the observer observes the subject eye. The first and second through-holes are situated diametrically opposite one another, and there is a further through-hole in the reflective means. Again, while this goes some way to reducing the expense of the opthalmoscope, it does not lend itself to mass production and is not simple to mount, requiring precision in the location of the three through-holes which must be aligned exactly.

### OBJECT OF THE INVENTION

The object of the present invention is to provide an opthalmoscope that is cheap and simple to manufacture, so that it can be produced in large quantities at relatively low cost, thus making it widely available to medical practitioners and students of medicine who would not normally be able to afford an opthalmoscope.

### SUMMARY OF THE INVENTION

An opthalmoscope according to the present invention comprises:

an attachment portion, for attaching an electromagnetic radiation source thereto;

an electromagnetic radiation deflector, for deflecting electromagnetic radiation from a first path into a second path;

a deflector housing, for holding the deflector in fixed relation to the attachment portion, so as to define the first path; and

a stenopeic aperture, in the first path, for limiting the electromagnetic radiation incident on the deflector to a specified meridian.

For preference, the stenopeic aperture is a slit formed in the housing, the longer axis of which lies in the plane formed between the first and second paths.

Preferably, the attachment portion is formed from a resilient material shaped to hold an electromagnetic radiation source in both slidable engagement and rotatable engagement, such that the deflector is moveable towards or away from the electromagnetic radiation source along the first path and is rotatable around an axis defined by that path.

For further preference, the housing is integral to the attachment portion, both being formed from a single sheet of resilient material.

More preferably still the deflector is one of a rear silvered mirror or a prism.

For still further preference an electromagnetic radiation condenser, comprising a planar spherical lens, is attached either to the housing, or to the external surface of the prism in the second path, for condensing electromagnetic radiation deflected into the second path.

### BRIEF DESCRIPTION OF THE DRAWINGS

The present invention will now be described in greater detail, by way of example, with reference to the accompanying drawings, in which:

FIG. 1 shows a front elevation of the opthalmoscope according to the present invention;

FIG. 2 shows a cross-section along line A-B in FIG. 1 of the opthalmoscope according to the present invention;

FIG. 3 shows a plan view as seen from below of the opthalmoscope according to the present invention;

FIG. 4 shows a plan view of a blank made of resilient material used to make the opthalmoscope according to the present invention;

FIG. 5 shows a front elevation of the opthalmoscope including a condensing lens according to the present invention;

FIG. 6 shows a cross-section along line A′-B′ in FIG. 5 of the opthalmoscope including a condensing lens according to the present invention;

FIG. 7 shows a plan view as seen from below of the opthalmoscope including a condensing lens according to the present invention;

FIG. 8 shows a front elevation of the opthalmoscope including a reference sight according to the present invention;

FIG. 9 shows a cross-section along line A″-B″ in FIG. 8 of the opthalmoscope including a reference sight according to the present invention;

FIG. 10 shows a plan view of a blank made of resilient material used to make the embodiment of the opthalmoscope shown in FIGS. 8 and 9 according to the present invention;

FIG. 11 shows a schematic diagram of a method of direct ophthalmic observation of the retina of an eye using the opthalmoscope of the present invention; and

FIG. 12 shows a schematic diagram of a method of indirect ophthalmic observation of the retina of an eye using the opthalmoscope of the present invention.

### DETAILED DESCRIPTION OF THE INVENTION

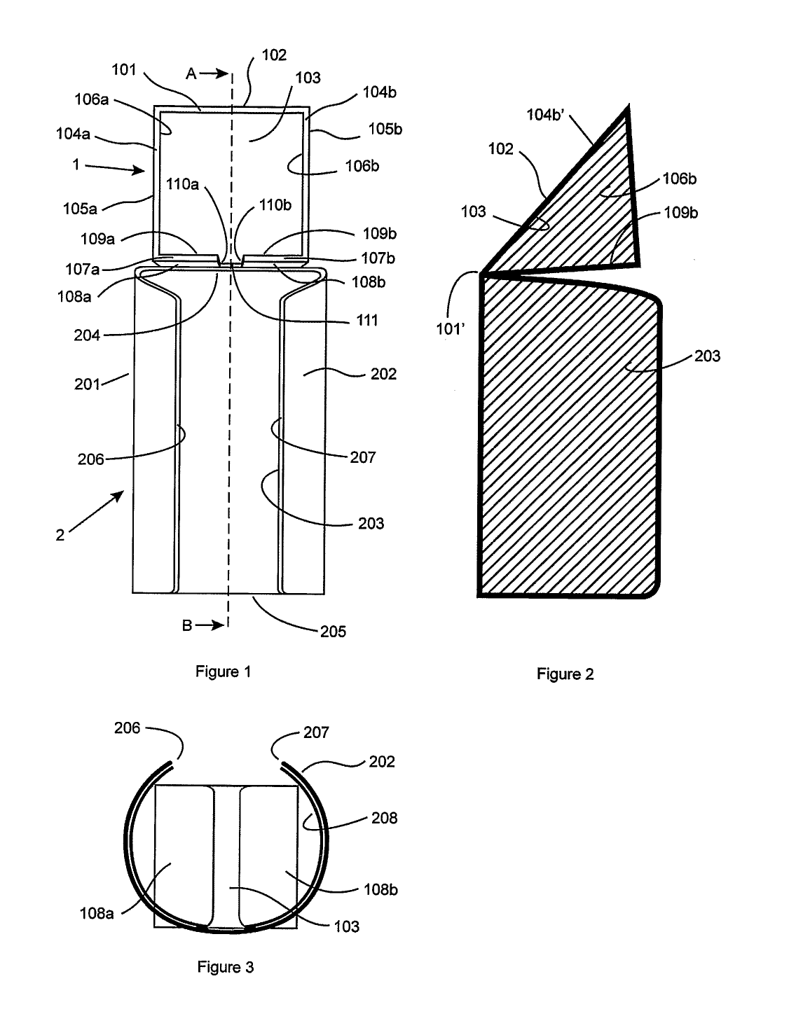

Referring first to FIG. 1 of the drawings, an opthalmoscope according to the present invention comprises an electromagnetic radiation deflector housing 1, connected to an electromagnetic radiation source attachment portion 2. Housing 1 comprises a deflector support wall 101 having an outer surface 102 and an inner deflector support surface 103. Deflector support surface 103 is bounded on two sides by two parallel side walls 104a and 104b, having respective edges 104a′ and 104b′ where they join with support wall 101. Side walls 104a and 104b each have respective outer surfaces 105a and 105b, and inner surfaces 106a and 106b, and extend, in the same direction, substantially perpendicularly from deflector support wall 101, such that edges 104a′ and 104b′ form the hypotenuse of a right-angled triangle as can be seen in FIG. 2. Edges 104a′ and 104b′ are chosen to have a length of between 16 mm and 22 mm, depending on the deflector configuration that is to be used, and support wall 101 has a width of 10 mm. The base and height of side walls 104a and 104b are chosen to be between 10 mm and 16 mm, also depending on the deflector configuration that is to be used. The above dimensions of support surface 103 are chosen depending upon the electromagnetic radiation deflector that is to be used. For preference a rear-silvered mirror (not shown) is attached to inner surface 103 of deflector support wall 101. Such a mirror has been found to work well with the opthalmoscope of the present invention, because it is not prone to fogging and, because it is not significantly absorbing in the visible electromagnetic radiation spectrum, does not significantly reduce the intensity of the electromagnetic radiation reflected there from. It is also possible to use a prism (not shown) instead of a mirror, in which case the sides of the prism are attached to inner surfaces 106a and 106b of side walls 104a and 104b. Housing 1 further comprises two aperture forming walls 107a and 107b, each having respective outer surfaces 108a and 108b, and inner surfaces 109a and 109b. Aperture forming walls 107a and 107b extend towards each other, substantially perpendicularly from respective side walls 104a and 104b on the base of the right-angled triangle. Aperture forming walls 107a and 107b have respective end faces 110a and 110b which lie substantially parallel to one another, and are separated from each other by a distance of between 1.5 mm and 2.0 mm, so as to form a stenopeic aperture 111. The stenopeic aperture shown in the drawing figures is a slit, but can be of any form, such as a circular or oval shaped orifice.

Referring to FIGS. 1 and 3 of the drawings, attachment portion 2 comprises a substantially semi-circular attachment wall 201 having an outer surface 202 and an inner surface 203. Attachment wall 201 is made from a resilient material, such as metal or plastic which can be easily formed into a semi-circle, and which is sufficiently yieldable to allow insertion of an electromagnetic radiation source therein, and is sufficiently resilient to grip the electromagnetic radiation source. Attachment wall 201 has a proximal end 204 and a distal end 205, wherein a part of said proximal end 204 is connected to deflector support wall 101 at hinge line 101′, such that the axis of rotation of attachment wall 201 passes through stenopeic slit 111. Attachment wall 201 also comprises sides 206 and 207 which are parallel to each other and are separated from one another sufficiently so as to allow attachment portion 2 to grip an electromagnetic radiation source inserted therein. Sides 206 and 207 are shorter than the distance between proximal end 204 and distal end 205 so as to allow housing 1 to be tilted towards an electromagnetic radiation source held in attachment portion 2. The diameter of semi-circular attachment wall 201, and the distance between the proximal end 204 and distal end 205 thereof is determined by the electromagnetic radiation source that is to be accommodated in attachment portion 2. In a preferred embodiment of the present invention, the electromagnetic radiation source is a commonly available penlight, and the diameter of semi-circular attachment wall 201 is therefore chosen to be approximately 10 mm, the distance between proximal end 204 and distal end 205 is chosen to be approximately 30 mm, and the length of sides 206 and 207 is chosen to be approximately 25 mm.

Referring to FIG. 3 of the drawings, inner surface 203 of attachment wall 201 is lined with a lining 208 so as to facilitate both rotation of an electromagnetic radiation source about the axis of rotation of semi-circular attachment wall 201, and translation of the electromagnetic radiation source towards or away from the electromagnetic radiation deflector held in housing 1. In the preferred embodiment of the present invention lining 208 is chosen to be felt, but may be any material that allows smooth rotational and translational movement of the electromagnetic radiation source.

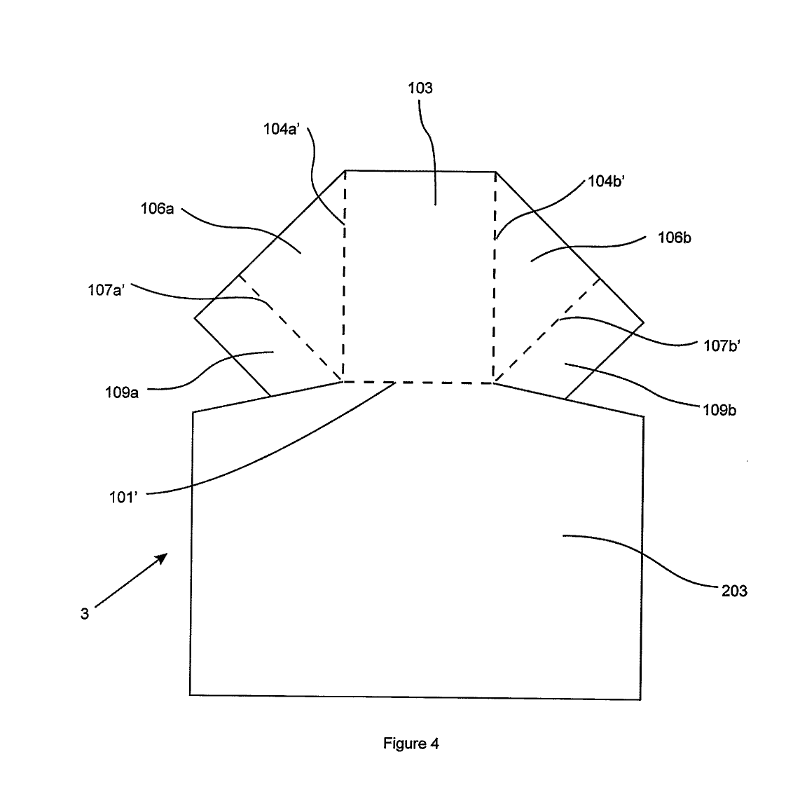

In order to reduce manufacturing costs it is preferable that housing 1 is integral with attachment portion 2, although it would be possible to make a separate housing 1 and attachment portion 2, and use a hinge or some other type of attachment means to attach one to the other. FIG. 4 shows a blank 3 for use in making the opthalmoscope according to the present invention showing the inner surfaces 103, 106a, 106b, 109a and 109b of housing 1, and inner surface 203 of attachment portion 2. Blank 3 may be of any resilient material, such as metal or plastic which is sufficiently malleable that it can be formed to create housing 1 and attachment portion 2, which is sufficiently yieldable to allow insertion of an electromagnetic radiation source into attachment portion 2 when formed, and allow tilting of housing 1 about hinge line 101′ toward and away from attachment portion 2, and which is sufficiently resilient to grip the electromagnetic radiation source. As can be seen from FIG. 4, blank 3 comprises the basic cut-out shape of both housing 1 and support portion 2, and includes score lines corresponding to edges 104a′ and 104b′ of support wall 101 with side walls 104a and 104b respectively, score lines corresponding to edges 107a′ and 107b′ of side walls 104a and 104b with aperture forming walls 107a and 107b respectively, and hinge line 101′ formed at the junction of proximal end 204 of attachment portion 2 with the lower edge of support wall 101 of housing 1.

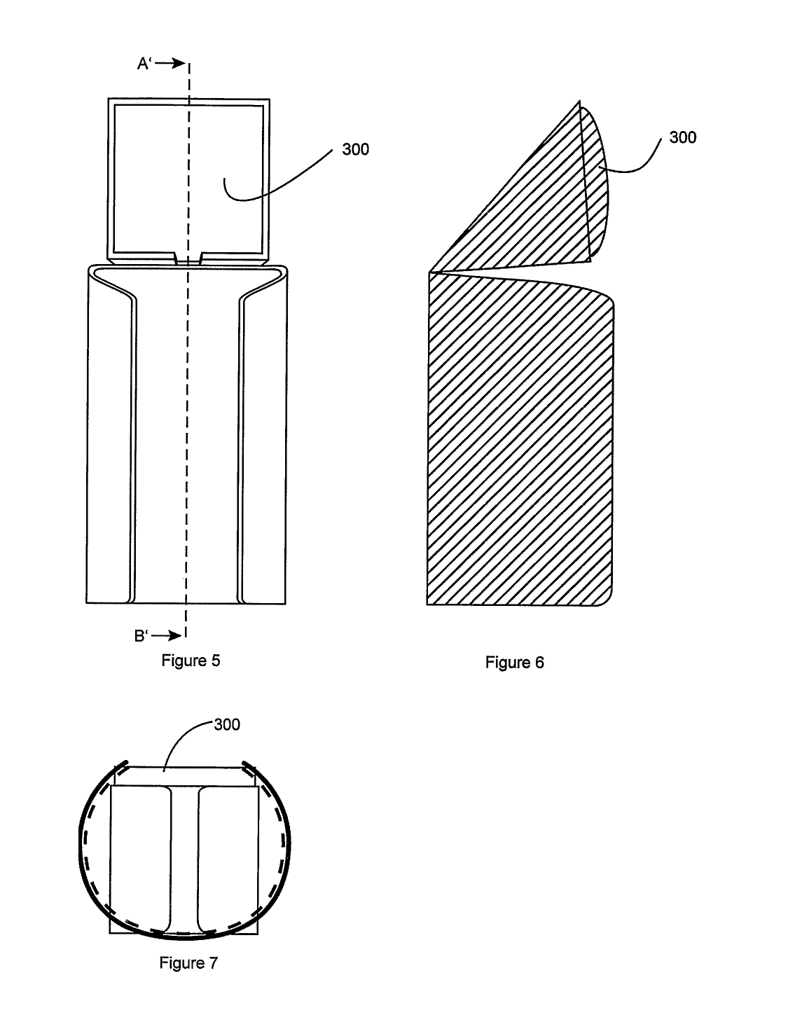

FIGS. 5, 6 and 7 show a further embodiment of the present invention where a prism is used as the electromagnetic radiation deflector, and the opthalmoscope is to be used to for indirect ophthalmic observation of an eye. Due to the fact that a prism absorbs radiation in the visible region of the electromagnetic spectrum, the intensity of the radiation exiting the opthalmoscope may, depending upon the electromagnetic radiation source, be insufficient to allow indirect ophthalmic observation of an eye. In this case, a condensing lens 300 is attached to the front of the prism as shown. This lens is chosen to be a simple planar spherical lens.

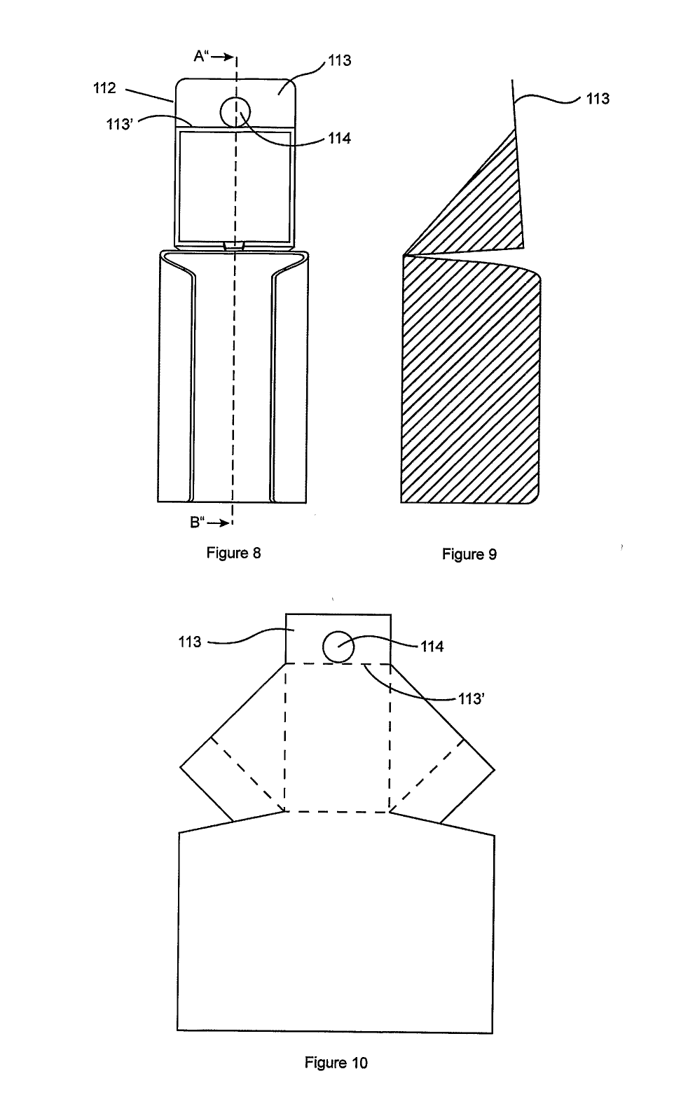

FIGS. 8 and 9 show a further embodiment of the present invention, including an observation sight 112 above the exit of the opthalmoscope. Observation sight 112 comprises a sight wall 113, extending above housing 1 from support wall 101 at an edge 113′. Sight wall 113 has an orifice 114 bored therein, the lower edge thereof touching edge 113′, and being centred thereon. The diameter of orifice 114 is chosen to be approximately 2.5 mm, and sight wall 113 extends a distance of approximately 5 mm above edge 113′.

FIG. 10 shows a blank 4 for use in making the opthalmoscope according to the present invention, including sight 112. Blank 4 is essentially the same as blank 3, with the addition of a sight wall 113 and including a score line at edge 113′ and orifice 114.

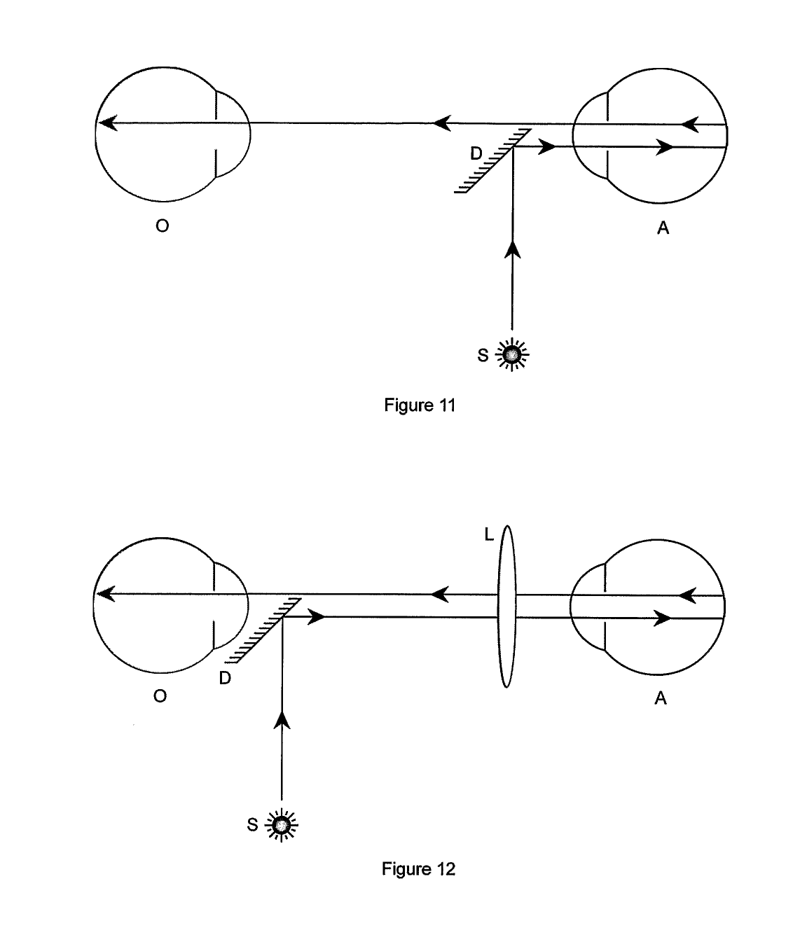

In its preferred embodiment, the opthalmoscope according to the present invention can be used to make both direct and indirect ophthalmic observations of the eye. FIG. 11 shows a schematic diagram of direct ophthalmic observation of the retina of an eye. In this case, a beam of light from source S is incident on deflector D which deflects the beam into subject eye A. The beam illuminates a relatively small area of the retina of subject eye A and reflected light passes over deflector D to observer eye O. The opthalmoscope is held close to subject eye A and the observer views a direct image of the retina. FIG. 12 shows a schematic diagram of indirect ophthalmic observation of the retina of an eye. In this case, a beam of light from source S is incident on deflector D which deflects the beam via a lens L into subject eye A. The beam illuminates a larger area of the retina of subject eye A, allowing the edge of the retina to be observed, and reflected light passes through lens L over deflector D to observer eye O. The opthalmoscope is held distant from subject eye A, close to observer eye O, and the observer views an inverted image of the retina. For indirect observation lens L is necessary to collimate light diverging from the opthalmoscope into the eye, in order to obtain an intensity of incident and reflected light sufficient for observation.

In order to use the opthalmoscope according to the present invention an electromagnetic radiation source must be held in attachment portion 2. As mentioned previously, it has been found that cheap, widely available, penlights are a suitable source of electromagnetic radiation and contribute to keep the cost of the opthalmoscope to a minimum. In the preferred embodiment of the present invention, where the electromagnetic radiation deflector is a rear-silvered mirror, a penlight is inserted into attachment portion 2, and is positioned so that the light beam there from passes through stenopeic aperture 111. Stenopeic aperture 111 is an essential component of the opthalmoscope according to the present invention, because it enables the aberrations in the light beam from the penlight to be reduced substantially, without reducing the intensity of the beam beyond that which is necessary for ophthalmic observation, giving a relatively aberration free beam, without the need for complicated optics and/or an expensive aberration free light source. Stenopeic aperture 111 works on the principle of astigmatic imagery, limiting the light rays incident on the electromagnetic radiation deflector to the meridian defined by the aperture.

With reference to FIGS. 1 and 11, when making a direct ophthalmic observation, the opthalmoscope according to the present invention is held close to the subject and light deflected from electromagnetic radiation deflector is directed into subject eye A. While in this position observer O is able to see light reflected from the retina of subject eye A, and may respectively reduce or increase the intensity of the light by moving the penlight in attachment portion 2 away from or toward housing 1. This has the effect of allowing less or more light from the penlight to pass through stenopeic aperture 111. In the event that the beam of light exiting the opthalmoscope contains too many aberrations, the observer can twist the electromagnetic radiation source so that it rotates about its axis and changes the position of the light beam with respect to stenopeic aperture 111, thereby enabling the most aberration free portion of the light beam to pass there through. This is particularly relevant where the electromagnetic radiation source is a penlight, because they contain coil filaments giving an elongated light beam. Also, because the system is astigmatic, the observer may easily vary the area of illumination of the retina by moving the opthalmoscope towards or away from subject eye A. Note also that, due to the fact that the opthalmoscope is made from a resilient material, housing 1 may be tilted about hinge line 101′ in relation to the penlight so that the light beam is incident at different positions along deflector support wall 101. This enables the observer to vary the position to that which is most comfortable for him or her.

With reference to FIGS. 1 and 12, when making an indirect ophthalmic observation, the opthalmoscope according to the present invention is held close to observer eye O and light deflected from electromagnetic radiation deflector is directed into subject eye A via lens L. Similarly to the situation for direct opthalmoscopy, the observer can vary the intensity of the light beam, choose the most aberration free beam and adjust the position of the beam along deflector support wall 101.

It should be observed that advantageous physical changes to the apparatus itself may be apparent to those skilled in the art, and as such, the scope of the present invention should be limited only by the terms and interpretation of the following claims.

1. An opthalmoscope characterised by comprising:
an attachment means for attaching an electromagnetic radiation source thereto;
a deflection means for deflecting electromagnetic radiation from a first path into a second path;
a housing adapted to hold said deflection means in fixed relation to said attachment means so as to define said first path; and
a stenopeic aperture means in said first path for limiting the electromagnetic radiation incident on said deflection means to a specified meridian.

2. An opthalmoscope according to claim 1, characterised in that said stenopeic aperture means is formed in said housing.

3. An opthalmoscope according to claim 1, characterised in that said stenopeic aperture means is a slit.

4. An opthalmoscope according to claim 3, characterised in that the longer axis of said slit lies in a plane formed between said first and second paths.

5. An opthalmoscope according to claim 1, characterised in that said attachment means is formed from a resilient material adapted to accommodate an electromagnetic radiation source in slidable engagement, such that said deflection means is moveable towards or away from the electromagnetic radiation source along said first path.

6. An opthalmoscope according to claim 1, characterised in that said attachment means is formed from a resilient material adapted to accommodate an electromagnetic radiation source in rotatable engagement, such that said deflection means is rotatable around an axis defined by said first path.

7. An opthalmoscope according to claim 1, characterised in that said housing is integral to said attachment means.

8. An opthalmoscope according to claim 7, characterised in that said housing and said attachment means are formed from a single sheet of resilient material.

9. An opthalmoscope according to claim 1, characterised in that said deflection means is a rear silvered mirror.

10. An opthalmoscope according to claim 1, characterised in that said deflection means is a prism.

11. An opthalmoscope according to claim 1, characterised by further comprising an electromagnetic radiation condensing means attached to said housing in said second path, for condensing electromagnetic radiation deflected into said second path.

12. An opthalmoscope according to claim 10, characterised by further comprising an electromagnetic radiation condensing means attached to the external surface of said prism in said second path, for condensing electromagnetic radiation deflected into said second path.

13. An opthalmoscope according to claim 11, characterised in that said condensing means is a planar spherical lens.

14. An opthalmoscope according to claim 12, characterised in that said condensing means is a planar spherical lens.
